(19) United States
(12) Patent Application Publication
(10) Pub. No.: US 2018/0134584 A1
(43) Pub. Date: May 17, 2018

(54) WORKING METHOD AND APPARATUS FOR MONITORING A UV DISINFECTION INSTALLATION

(71) Applicant: HYTECON AG, Luzern (CH)

(72) Inventors: ANDREAS KOLCH, HERFORD (DE); HONGBIN XU, HAMBURG (DE)

(21) Appl. No.: 15/814,448

(22) Filed: Nov. 16, 2017

(30) Foreign Application Priority Data
Nov. 17, 2016 (DE) 102016122075.2

(51) Int. Cl. C02F 1/32

(52) U.S. Cl. CPC C02F 1/325; C02F 2201/3222; C02F 2303/04

(57) ABSTRACT

A working method and an apparatus of a UV disinfection installation contains a water-flooded reactor chamber and at least one UV radiation source. The UV radiation source is arranged such that it can radiate in the reactor chamber. A UV sensor and an evaluation unit for the sensor signal are provided. The apparatus attains a high cleaning performance with little constructional engineering complexity. The cleaning performance is able to be detected exactly for the entire irradiated flow volume, and which is attained by virtue of the radiation source consisting of UV LEDs which are at least arranged on one side of the reactor chamber and by virtue of the UV sensor is arranged on the same side between the UV LEDs and by virtue of at least the inner surface of the reactor chamber lying opposite this side being embodied as a reflection surface for the UV light.

### CROSS-REFERENCE TO RELATED APPLICATION

This application claims the benefit, under 35 U.S.C. § 119, of German patent application DE 10 2016 122 075.2, filed Nov. 17, 2016; the prior application is herewith incorporated by reference in its entirety.

### BACKGROUND OF THE INVENTION

### Field of the Invention

The invention relates to a working method and an apparatus for monitoring a ultraviolet (UV) disinfection installation having a water-flooded reactor chamber and at least one UV radiation source, which is arranged such that it can radiate therein, and having a UV sensor and an evaluation unit for the sensor signal.

Such an apparatus is already known from U.S. patent publication No. 2003/0170151 A1. Here, a multiplicity of LEDs with a planar arrangement extends in the flow of a liquid, wherein sensors are arranged in a few regions of the housing. The sensors detecting the current state values, in each case in the corresponding region, and transmitting these to an evaluation unit.

A disadvantage of this prior system is that only a small part of the volumetric flow of a liquid can be detected, and so a plurality of sensors has to be arranged along the length of the apparatus in order to obtain a meaningful result. However, it is known that a measurement is only carried out at a “representative” location, and so a completely correct statement about the actually attained cleaning performance cannot be made by any means.

### SUMMARY OF THE INVENTION

It is an object of the invention to provide a working method and an apparatus of a UV disinfection installation, which attain a high cleaning performance with little constructional engineering complexity, said cleaning performance being able to be detected exactly for the entire irradiated flow volume.

What is achieved as a result of the apparatus for monitoring a UV disinfection installation made of a water-flooded reactor chamber and at least one UV radiation source, which is arranged such that it can radiate therein, a UV sensor, and an evaluation unit for the signal being equipped, in which the radiation source consists of UV LEDs which are arranged on one side of the reactor chamber and in which the UV sensor is arranged on the same side between the UV LEDs and in which at least the inner surface of the reactor chamber lying opposite this side is embodied as a reflection surface for the UV light, is that the UV radiation has passed through the reactor chamber at least twice between being radiated into the reactor chamber and the incidence on the UV sensor since the UV radiation, after reaching the base of the reactor chamber, is reflected back to the sensor from there, as a result of which the measurement region is twice as long as the housing of the reactor chamber is deep and the sensor signal is qualitatively improved.

Using such a UV sensor it is possible to capture e.g. such parameters, like the power of the UV LEDs or possibly a reduction of same, caused by aging processes or an attenuation of the UV radiation by the formation of a lining on the water-facing side or else by particulate substances that are dissolved in the water.

Advantageous configurations of the subject matter of the invention emerge with and in combination from the subsequent dependent claims.

The UV LEDs, which are arranged next to the UV sensor, are preferably arranged on a carrier module and can easily be aligned in terms of their number and performance depending on the required cleaning and disinfection performance, as a result of which the apparatus is able to cover a large power spectrum or, moreover, work with different wavelengths of the UV LEDs by an optional selection of carrier modules.

According to a particularly preferred embodiment of the invention, the carrier module or the carrier modules are situated outside the water-flooded reactor chamber, with the UV LEDs lying directly on a quartz window, the side of which facing away from the LEDs forming a reactor inner wall and the upper border of the reactor chamber such that the UV LEDs do not come into contact with the liquid to be disinfected but do not experience any damping in their power by way of the quartz window either. Since the LEDs moreover remain cold to all practical purposes on the side facing the water during operation, the quartz window is not heated either, and so there is no need to be apprehensive about the formation of a lining thereon, as is often the case with reactors that operate with gas-discharge lamps as UV light sources.

A particular advantage of the present apparatus consists of the UV LEDs being placed and aligned in such a way that a uniform homogeneous UV radiation field made of UV radiation that is reflected at least once, but also reflected multiple times on the side faces of the reactor space, is produced in the reactor chamber such that the UV sensor can detect the virtually uniform diffuse radiation field that is prevalent in the reactor chamber substantially more accurately and it is possible, for example, to recognize possible changes in the UV transmission with an extraordinary accuracy, with the UV sensor preferably being aligned on a spatially specific sensor region of the homogeneous UV radiation field, as a result of which the detected sensor signal of the sensor region is extrapolatable over the whole homogeneous UV radiation field by way of the evaluation unit and the result of the extrapolation reflects a highly accurate image of the actually obtained disinfection result on account of the homogeneity of the UV radiation field.

Other features which are considered as characteristic for the invention are set forth in the appended claims.

Although the invention is illustrated and described herein as embodied in a working method and an apparatus for monitoring a UV disinfection installation, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic, plan view of a UV disinfection installation; and

FIG. 2 is a sectional view of the UV disinfection installation according to FIG. 1.

### DETAILED DESCRIPTION OF THE INVENTION

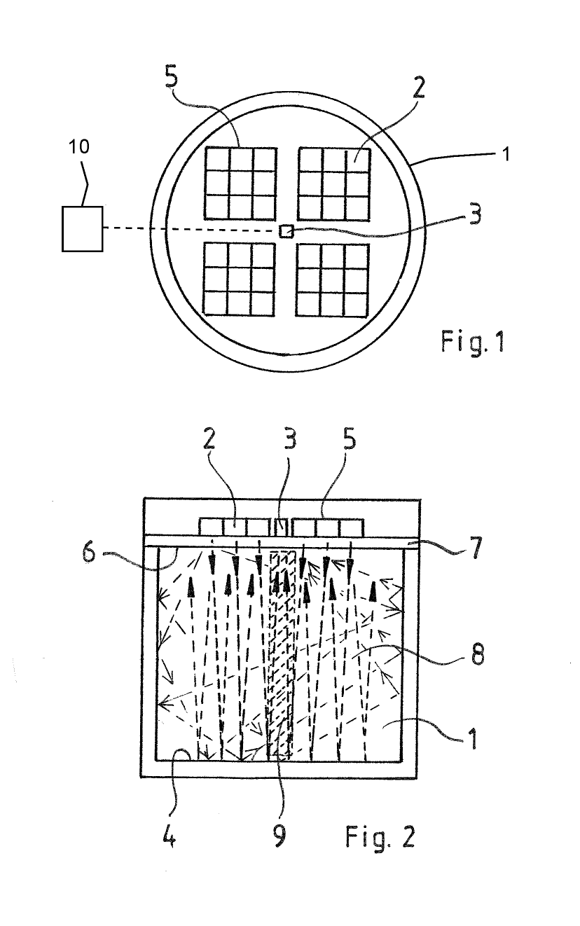

Referring now to the figures of the drawings in detail and first, particularly to FIGS. 1 and 2 thereof, there is shown a UV disinfection installation that consists of a water- or liquid-flooded reactor chamber 1 that is covered by a quartz window 7 through which UV LEDs 2 radiate their UV light into the reactor chamber 1. The UV light is reflected on a surface that is embodied as a reflection surface 4 and that lies opposite the quartz window 7 such that a UV sensor 3 which is arranged adjacent to the UV LEDs 2 is able to detect the UV light after two passages through the reactor chamber 1. The detected values are transmitted to an evaluation unit 10 and being prepared and evaluated by the evaluation unit 10.

A homogeneous UV radiation field 8 is produced in the reactor chamber 1, with this not necessarily having to be assumed to be an approximate parallel to-and-fro radiation of the UV light, as sketched only approximately in FIG. 2, since a strongly diffuse radiation may be prevalent in the reactor chamber 1 on account of the single or multiple reflection of the UV light therein on the lateral reflection surfaces of the reactor chamber 1. The strongly diffuse radiation nevertheless being largely homogeneous such that the sensor region 9 that is captured by the UV sensor 3, or the detected values therefrom, can still be extrapolated over the volume of the entire reactor chamber 1 in order to generate exact values for the entire volume, just as it is possible to detect, by way of the evaluation unit, changes in the UV transmission by substances that are dissolved in the water or by particulate substances or, for example, the formation of a lining on the side of the quartz window 7 that faces the water.

1. A self-monitoring ultraviolet (UV) disinfection installation, comprising:
a water-flooded reactor chamber having a first side and an opposite second side;
at least one ultraviolet (UV) radiation source disposed to radiate in said water-flooded reactor chamber, said at least one UV radiation source having UV light emitting diodes (LEDs) disposed on said first side of said water-flooded reactor chamber;
an UV sensor outputting a sensor signal and disposed on said first side between said UV LEDs;
an evaluation unit receiving the sensor signal; and
said water-flooded reactor chamber having on at least an inner surface of said second side a reflection surface for UV light.

2. The UV disinfection installation according to claim 1, wherein said at least one ultraviolet (UV) radiation source has a carrier module and said UV LEDs are disposed on said carrier module and in that a plurality of said UV LEDs is brought in line with a required cleaning and disinfection performance.

3. The UV disinfection installation according to claim 2,
further comprising a quartz window; and
wherein said carrier module is situated outside of said water-flooded reactor chamber and said UV LEDs lie directly on said quartz window, a side of said quartz window facing away from said UV LEDs forming a reactor inner wall and an upper border of said water-flooded reactor chamber.

4. The UV disinfection installation according to claim 1, wherein said UV LEDs are placed and aligned in such a way that a uniform homogeneous UV radiation field is radiable into said water-flooded reactor chamber.

5. The UV disinfection installation according to claim 4, wherein said UV sensor is aligned on a spatially specific sensor region of the uniform homogeneous UV radiation field.

6. The UV disinfection installation according to claim 5, wherein a detected sensor signal of the spatially specific sensor region is extrapolatable over a whole of the uniform homogeneous UV radiation field by way of said evaluation unit.

7. A working method for monitoring a UV disinfection installation, by means of an apparatus, which comprises the steps of:
producing, via ultraviolet (UV) LEDs, a uniform homogeneous UV radiation field in a reactor chamber; and
capturing, in a spatially restricted sensor region, via an UV sensor, UV light radiated into the reactor chamber by the UV LEDs after the UV light has passed through the reactor chamber at least twice.

8. The working method according to claim 7, which further comprises:
receiving in an evaluation unit, a sensor signal of the UV sensor; and
proceeding from values detected in the spatially restricted sensor region, extrapolating the values over the uniform homogeneous UV radiation field entirely.

9. The working method according to claim 8, wherein the evaluation unit detects possible changes in a UV transmission.

10. The working method according to claim 9, which further comprises detecting, via the evaluation unit, a formation of a lining on a side of a quartz window that faces water and/or an attenuation of the UV transmission by substances that are dissolved in the water or by particulate substances.
